(19) United States
(12) Patent Application Publication
(10) Pub. No.: US 2019/0151154 A1
(43) Pub. Date: May 23, 2019

(54) MATERIALS REDUCING FORMATION OF HYPOCHLORITE

(71) Applicant: Sumitomo Chemical Company Limited, Tokyo (JP)

(72) Inventors: Christopher Kay, Godmanchester (GB); Thomas Kugler, Godmanchester (GM); Sheena Zuberi, Godmanchester (GB)

(73) Assignee: Sumitomo Chemical Company Limited, Tokyo (JP)

(21) Appl. No.: 16/193,878

(22) Filed: Nov. 16, 2018

(30) Foreign Application Priority Data
Nov. 17, 2017 (GB) 1719034.9

(51) Int. Cl. A61F 13/00; A61L 15/42; A61L 15/18; A61L 15/20; B01J 31/22

(52) U.S. Cl. CPC A61F 13/00063; A61L 15/42; A61L 15/18; A61L 2300/224; B01J 31/2273; A61L 2300/102; A61L 15/20

(57) ABSTRACT

The invention relates to electrochemical wound dressings wherein potential damage to healthy cells and granulating tissue induced by hypochlorite and/or hypochlorous acid is reduced by enabling effective removal or build-up of substantial concentrations thereof through use of catalysts and/or scavenging/sacrificing agents. In another aspect, the present invention relates to the use of the latter materials in medical devices and articles.

### RELATED APPLICATIONS

This application claims foreign priority benefits under 35 U.S.C. § 119(a)-(d) or 35 U.S.C. § 365(b) of British application number 1719034.9, filed Nov. 17, 2017, the entirety of which is incorporated herein.

### FIELD OF INVENTION

This invention relates to an electrochemical wound dressing comprising materials for preventing formation of hypochlorite or for conversion of chlorine (a precursor of hypochlorite) or hypochlorite into species with improved biological acceptability.

In further aspects, the present invention relates to the use of said materials in medical devices or articles.

### BACKGROUND OF THE INVENTION

In the recent years, there has been an increased interest in the development electrochemical medical devices and articles for a growing number of applications.

For example, WO 2004/049937 A1 and WO 2005/099644 disclose wound dressings adapted to monitor specific wound conditions through measurements of electrical properties or characteristics.

US 2008/0288019 A1 discloses neurostimulation devices for pain treatment and therapy that are configured to promote the electrochemical generation of oxidants.

WO 2017/011635 A1 discloses dressings for the treatment of open wounds, wherein hydrogen peroxide is continuously produced via an electrochemical reaction of oxygen in air and water at the wound surface in order to reduce or remove biofilms which may represent a diffusion barrier to antibiotics.

However, electrochemical medical devices (such as electrochemical wound dressings or bandages, for example) being in contact with aqueous electrolyte solutions containing chloride ions present the risk of generating hypochlorite anions (ClO⁻) and its protonated form, hypochlorous acid (HClO), which may potentially be detrimental in biomedical systems, specifically in those designed for the treatment of chronic wounds.

Specifically, while sodium hypochlorite (a component of Dakin's solution) is known to have a bactericidal effect against most organisms commonly found in open wounds and has been widely used in the treatment of pressure ulcers with necrotic tissue in order to help control infection, it is cytotoxic to healthy cells and granulating tissues at concentrations of 0.25% or higher (≥3.4 mM; see J.P. Heggers, Journal of Burn Care and Rehabilitation 1991, 12(5), 420-424). Specifically, ClO⁻ and HClO are critical reactive oxygen species (ROS) in biological and biomedical systems, and their uncontrolled production may potentially lead to tissue damage (e.g. local necrosis) and diseases, including renal failure (see e.g. B.W. Peck et al., Saudi J. Kidney Dis. Transpl. 2014, 25(2) 381-384) and arthritis (see e.g. Immunopathogenetic Mechanisms of Arthritis; J. A. Goodacre, G. Dick; MTP Press 1986, Norwell, USA).

In electrochemical medical systems, hydroxide anions produced at the cathode (with hydrogen gas as a byproduct) react with the chlorine generated at the anode to produce ClO⁻/HClO and oxygen as a byproduct according to the following reactions:

2Cl⁻→Cl₂+2e⁻ Anode:

2H₂O+2e⁻→H₂+2OH⁻ Cathode:

Cl₂+2OH⁻→Cl⁻ClO⁻+H₂O Hypochlorite Formation:

NaCl+H₂O→NaClO+H₂

Whereas WO 2017/011635 A1 discloses an embodiment which enables electrochemical HOCl generation by converting chlorine and water, means for effective conversion and/or formation prevention of hypochlorite in medical devices have hitherto not been addressed.

In view of the above, there exists a need for electrochemical medical devices which reduce the potential damage to healthy cells and granulating tissue induced by reactive oxygen species by enabling effective removal or build-up of substantial concentrations of hypochlorite and/or hypochlorous acid.

### SUMMARY OF THE INVENTION

The present invention solves these objects with the subject matter of the claims as defined herein. The advantages of the present invention will be further explained in detail in the section below and further advantages will become apparent to the skilled artisan upon consideration of the invention disclosure.

The present inventors found that by implementing materials that catalytically convert Cl₂, OCl⁻ and/or HClO to biologically compatible species and/or materials which may react as a substitute for the precursor species (such as e.g. chloride) leading to the generation of Cl₂, OCl⁻ or HClO, and/or reduce OCl⁻/HClO into electrochemical devices or articles, the generation of substantial concentrations of Cl₂, OCl⁻ and/or HClO from the electrochemical conversion of chloride ions may be suppressed effectively and in a simple manner. Accordingly, electrochemical medical systems may be manufactured which enable safe use for a prolonged period of time (e.g. for treatment of chronic wounds) without the risk of potential cytotoxic effects originating from OCl⁻/HClO (and resulting tissue damages, potential diseases etc.) and which promote and/or accelerate wound healing.

In a first aspect, the present invention relates to an electrochemical wound dressing, comprising: first and second electrodes, an electrical power source in electrical contact with the first and the second electrode, and at least one material selected from a first material and a second material; wherein the first material is a catalyst for the electrochemical reduction of hypochlorite anions or hypochlorous acid, and wherein the second material is capable of reacting as a substitute for the precursor species leading to the generation of hypochlorite anions or hypochlorous acid and/or capable of reducing hypochlorite anions or hypochlorous acid.

In a second aspect, the present invention relates to use of at least one material selected from the first and second materials described above for preventing formation of or for reducing hypochlorite anions or hypochlorous acid in a medical device or article.

Preferred embodiments of the formulation according to the present invention and other aspects of the present invention are described in the following description and the claims.

### BRIEF DESCRIPTION OF THE DRAWINGS

FIG. 1 illustrates reactions occurring at the anode and cathode of an electrochemical system using aqueous solutions containing chloride ions as electrolytes.

FIG. 2A illustrates an exemplary wound dressing of the present invention from below (wound side).

FIG. 2B shows the side view of an exemplary wound dressing of the present invention.

### DETAILED DESCRIPTION OF THE INVENTION

For a more complete understanding of the present invention, reference is now made to the following description of the illustrative embodiments thereof:

### Electrochemical Wound Dressing

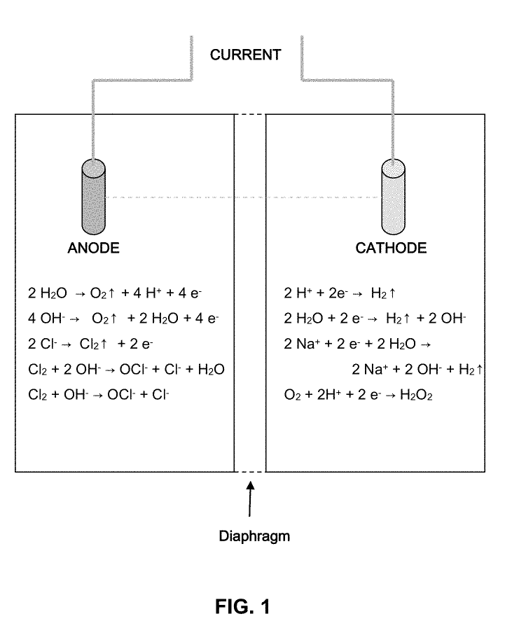

Hypochlorite anions or hypochlorous acid can diffuse through cells and react with amino groups to form chloramines, which persist as long-lived oxidants, hence, release of hypochlorite, particularly at inflammatory sites, may have severe detrimental effects on the well-being of the patient and the wound healing process. Electrochemical medical devices which involve the use of aqueous solutions containing chloride ions present the risk of generating Cl₂, ClO⁻ or HClO through oxidation processes as shown in FIG. 1.

In a first embodiment, the present invention therefore relates to an electrochemical wound dressing, comprising: first and second electrodes, an electrical power source in electrical contact with the first and the second electrode, and at least one material selected from a first material and a second material; wherein the first material is a catalyst for the electrochemical conversion of hypochlorite anions or hypochlorous acid, and wherein the second material is capable of reacting as a substitute for the precursor species leading to the generation of hypochlorite anions or hypochlorous acid and/or capable of reducing hypochlorite anions or hypochlorous acid. Said embodiment minimizes or prevents the formation of ClO⁻/HClO⁻ at the wound site and simultaneously allows to make use of the bactericidal effects of hydrogen peroxide formed at the electrode acting as a cathode.

The term “dressing”, as used herein, refers to a sterile article that may include a bandage, adhesive tape, a sterile pad, compress, pack, gauze, or mat, for example, and that can be applied to open wounds for staunching bleeding, absorbing exudate, easing pain, debriding the wound, reducing or preventing infections, or promoting healing. The dressing may comprise multiple layers to enhance patient comfort and promote healing, including layers of fabric, cotton gauze, poly(ethylene glycol)-water, poly(ethylene oxide)-water polymer, as well as layer(s) containing topical ointments and biologically active components, including antibiotics, growth factors, and antiseptics, for example.

The term “open wound” generally refers to an injury to a human or animal body causing damages to epidermis, dermis, cutaneous tissues and/or subcutaneous tissues of the human or animal body.

The expression “chronic wounds”, as used herein, refer to wounds that fail to proceed through the normal phases of wound healing in an orderly and timely manner since they stall in the inflammatory phase of wound healing and are ones which frequently have a strong tendency to recur.

The first material is not particularly limited as long as it catalyzes the electrochemical reduction of hypochlorite/hypochlorous acid (Cl oxidation state:+1; O oxidation state:−2), typically into chloride ions (Cl oxidation state:−1), during which process oxygen may be oxidized (to O₂) or maintain his oxidation state (in terms of H₂O as a product, for example). In a preferred embodiment, the first material is selected from a metal, a metal oxide, an organometallic complex, and mixtures thereof. As examples thereof, metals from any of groups 4 to 12, preferably from any of groups 4 and 8 to 11, or alkaline earth metals may be mentioned. In a further preferred embodiment, the first material comprises one or more metals selected from Mg, Ca, Ti, Mo, Mn, Fe, Ru, Co, Rh, Ir, Ni, Pd, Pt, and Cu, their oxides, complexes thereof, and mixtures of the aforementioned. As bimetallic catalysts, Rh—Cu and Rh—Ru may be mentioned. Preferred mixtures include mixtures of cobalt oxide with molybdenum oxide and/or nickel oxide, or substituted spinel-type cobalt oxides (as e.g. disclosed in U.S. Pat. No. 4,442,227 A), for example. Especially preferred are Mg, Ti, Mn, Fe, Co, Ni and Cu, their oxides and/or complexes, and mixtures of the aforementioned. Examples of organometallic complexes include, but are not limited to, cobalt porphyrin complexes, such as vitamin B12, for example.

Preferably, the first material is comprised in a porous catalyst layer in direct contact with the first or the second electrode, or in a membrane-like catalyst layer between the first and the second electrode to prevent bulk mixing. In any case, the catalyst layer can be encapsulated in a liquid-permeable membrane to prevent direct contact with the wound.

The second material is not particularly limited as long as it is capable of reacting as a substitute for the precursor species (e.g. chloride or chlorine) leading to the generation of hypochlorite anions or hypochlorous acid (e.g. a sacrificial species that can be oxidized in preference to the chloride ions) and/or capable of reducing hypochlorite anions or hypochlorous acid. The precursor species is typically chloride.

In a preferred embodiment, the second material is selected from small-molecule antioxidants including, but not limited to, ascorbic acid, sodium ascorbate, methionine, ammonium salts, taurine, lycopene, chlorogenic acid, lipoic acid, thiols, superoxide dismutase mimetics (mSOD), vitamin E, gallic acid, catechin, and polymers derived therefrom (e.g. conjugates of the aforementioned small-molecule antioxidants to high molecular weight poly(ethylene) (UHMPE), poly(acrylic acid), gelatin, poly(methyl methacrylate) and poly(ethylene glycol) or co-polymers wherein antioxidants are embedded into the backbone, such as poly(1,8-octanediol-co-citrate-co-ascorbate) (POCA)). Further preferred examples of the second material include ascorbic acid, sodium ascorbate, and polymers or co-polymers derived therefrom, methionine, lycopene, chlorogenic acid, lipoic acid and thiols. Cysteine, D-penicillamine, tiopronin (N-[2-mercaptopropionyl]glycine), sodium aurothiomalate, glutathione, or aurothioglucose may be mentioned as exemplary thiols. Exemplary reactions of ascorbic acid and ascorbate with hypochlorite are shown in the following schemes:

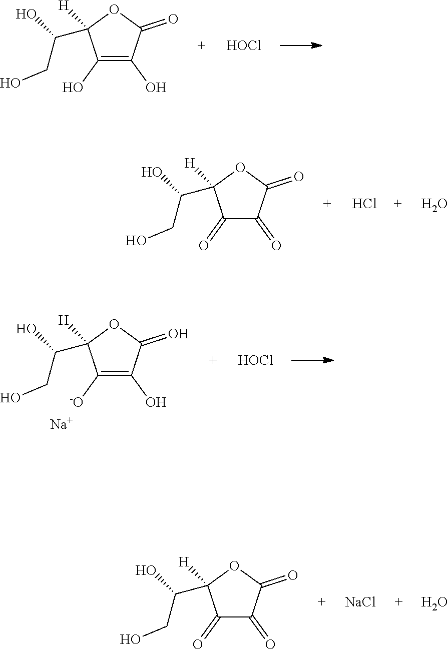

Incorporating the above-described second material in a layer in contact with the electrode may provide the additional benefit that the electrode then behaves like a reference electrode, i.e. as a “non-polarizable electrode”, which maintains a relatively constant potential despite the passage of current (as is the case with a conventional Ag/AgCl reference electrode, for example).

In further embodiments, the dressing may further include one or more power sources, voltage/current controllers, voltage/current sensors, and/or other suitable electrical/mechanical components. Preferably, the dressing comprises a constant current control unit between and in electrical contact with the electrical power source and the first and second electrodes. In yet further embodiments, the dressing may comprise a sensor detecting the concentration of hypochlorite/hypochlorous acid or precursor species thereof, coupled with a regulating means configured to switch the power source off/on or to reduce/increase the power output in case the sensed concentration is over/under a predetermined value.

The first and second electrodes may be electrically coupled to first and second polarities of a power source via an optional switch. The electrical power source is not particularly limited and may be embedded into the dressing structure or may be provided externally (e.g. as a kit of components) with suitable connection means. In one embodiment, the power source can include a thin-film battery having a target voltage differential of at least 1.2 V (e.g., about 1.4 to 1.6 V) and an areal capacity of 2 mAh/cm² or more (e.g. about 4 to 5 mAh/cm²). Suitable batteries include flexible and non-flexible, disposable and rechargeable batteries conventionally used in smart cards, skin patches, RFIDs, wearables, E-textiles, medical devices and consumer electronics, and may be appropriately selected by the skilled artisan depending on the dimensions of the wound or dressing and the desired application and performance. Preferably, the electrical power source is a flexible battery and/or a polymer battery. The electrochemical wound dressing may optionally further comprise a battery state indicator, e.g. integrated into a constant current controller. In other embodiments, the power source can also include a solar cell, a fuel cell, or other suitable direct current power sources.

The electrical interconnects between the electrical power source and the first and the second electrodes may be embedded in an electrically insulating layer, which may be made of or comprise electrically insulating polymer material, for example. Both the first and second electrodes may be made of or comprise electrically conductive materials commonly used for this purpose. Exemplary materials include, but are not limited to carbon (including, but not limited to graphite, carbon nanotubes, carbon particles dispersed in a non-conjugated polymer matrix), aluminum, silver, platinum, gold, palladium, tungsten, indium, zinc, copper, nickel, iron, stainless steel, oxides thereof, and combinations of the aforementioned. The electrodes may be encapsulated in or comprise a conductive material or salt to either ensure good adhesion or improved conduction. In a preferred embodiment, the wound dressing comprises a conductive polymer layer over and in contact with either the first or the second electrode. Suitable conductive polymers include, but are not limited to electrically conductive polymers or combinations of polymers selected from polyfluorenes, polyphenylenes, polypyrenes, polyazulenes, polynaphthalenes, polypyrroles, polycarbazoles, polyindoles, polyazepines, polyanilines, polythiophenes, polyacetylenes, and copolymers thereof. Preferably, the electrically conductive polymer is selected from conductive species based on poly(3,4-ethylenedioxythiophene) (PEDOT), polyaniline (PANI), polypyrrole (PPy), poly(phenylene vinylene) (PPV), poly(arylene), polyspirobifluorene, poly(3-hexylthiophene) (P3HT), poly(o-methoxyaniline) (POMA), poly(o-phenylenediamine) (PPD), or poly(p-phenylene sulfide), which may also include functionalized PEDOT with additional functional groups on the end of the polymer chain (e.g., PEDOT-TMA (PTMA), PEDOT-PEG, and PEDOT block PEG) or PEDOT with additional functional groups on the ethylenedioxy units. As an especially preferred conductive polymer, poly(3,4-ethylenedioxythiophene): polystyrene sulfonate (PEDOT:PSS) may be mentioned, as it is may be easily deposited by solution processing methods (e.g., by spin coating or electrochemical deposition) and exhibits favourable electrical properties (e.g. good electrical and ionic conductivity).

Preferably, the conductive layer comprising conductive materials, such as the above-described conductive polymers, is provided by electrochemical deposition methods or by solution processing methods, which enables simple and inexpensive production of the wound dressing.

In embodiments, a hydrogel layer may be provided between the conductive layer and the wound, which keeps the wound moist and thereby facilitates healing. The term “hydrogel”, as used herein, includes a coherent, three-dimensional aqueous polymer system capable of imbibing water without liquefying. In embodiments, insolubility in water may be provided by crosslinking of a hydrogel polymer. Examples of biologically acceptable hydrogels include, but are not limited to hydrogels based on (modified) collagen, (modified) dextran, agarose, gellum gum, poly (hydroxyalkyl methacrylates), poly (ethylene glycol), poly (propylene glycol), poly (acrylamide), poly (methacrylamides), poly (vinyl alcohol), poly (N-vinyl pyrrolidone), and derivatives thereof; as well as anionic and cationic hydrogels. By suitably adjusting the water content, gelling agents, buffering capacity and pH, the wound care effect may be further improved. Also, salts may be preferably added to the hydrogel to enhance its ionic conductivity.

In a preferred embodiment, the first electrode is configured as a peripheral wound electrode (or a so-called “peri-wound electrode”, i.e. an electrode being in contact with peri-wound skin upon application of the dressing) and the second electrode is configured as a wound electrode (i.e. an electrode being positioned over the wound upon application of the dressing). In such a configuration, the second material is preferably comprised in the peripheral wound electrode (which may function as the anode, for example) and/or a conductive layer is applied on the wound electrode (which may function as the cathode, for example).

In embodiments, the dressing may also include an aqueous solution (e.g., an aqueous solution comprising organic or inorganic salts (e.g. buffer solutions based on phosphates, citric acid, (bi)carbonates, acetate, HEPES, MES, TRIS, etc.) and/or antibiotic solution), for example in an absorbent material or another reservoir, which may be continuously or periodically provided to the wound to maintain a moist environment at the wound surface.

The dressing may include a substrate comprising one or more layers, on which the above-described materials or layers are provided. Suitable materials for substrates may be appropriately selected by the skilled artisan and include, but are not limited to fabric (e.g. woven fabric), cotton gauze, polymers (including vinyl, polyalkylenes (e.g. polypropylene, polyethylene), polyethylene terephthalate, rubbers (e.g. latex), copolymers, silicones, polyester-, polyurethane-, polystyrene-, EVA (ethylene vinyl acetate)-, or polyamide-based materials, for example) and bioabsorbable or biocompatible materials.

It will be understood that the dressing may further comprise adhesive layers commonly used in the art, for lamination of each of the above-described layers and/or for secure attachment of the dressing onto the skin.

The thicknesses of each of the above-described components and layers are not particularly limited and may be suitably selected from the skilled artisan depending on the application, the dimensions of the wound and/or dressing, and the desired mechanical properties (e.g., elasticity or rigidity) of the resulting dressing.

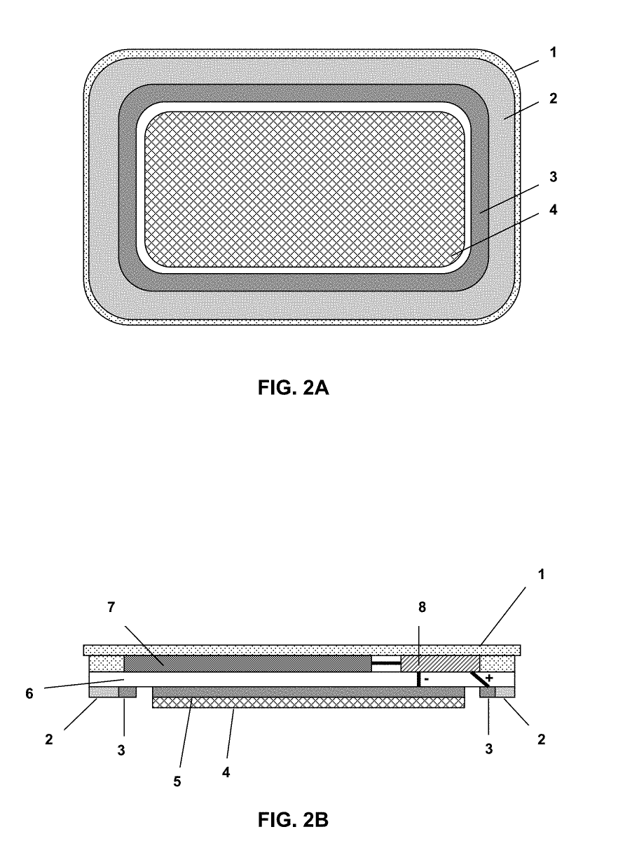

An exemplary electrochemical wound dressing according to the present invention which comprises the second material explained above is illustrated in FIGS. 2A and 2B. Herein, a power source (7) (e.g. a flexible battery with a voltage output of 1.5 V and an areal capacity of 4.5 mAh/cm²) is provided in on a substrate (1) in electrical contact with an optional constant current control unit (8) (which may control the current to a set value in a range of 5 to 30 μA, for example). An electrically insulating backing layer (6) is provided over the power source (7) and the constant current control unit (8), with electrical interconnects between the positive/negative polarity of the unit (8) and the first/second electrode (3)/(5) being embedded therein. The first electrode (3) comprises a second material capable of reacting as a substitute for the precursor species leading to the generation of hypochlorite anions or hypochlorous acid and/or capable of reducing hypochlorite anions or hypochlorous acid, and is configured as a peripheral wound electrode (i.e. a peripheral wound anode), surrounded by an adhesive layer (2). The second electrode (5) is configured as an electrode (i.e. cathode) to be positioned over the wound surface, with a conductive layer (4) formed between the second electrode and the wound, wherein an optional hydrogel layer (not shown) may act as a bridging layer between the wound and the conductive layer (4). The first and second electrodes are spaced apart from each other, optionally with an insulating material provided in between (not shown in the figures). The exemplary dressing is configured so that the circuit is completed by hydration between the first electrode (3) and the second electrode (5), enabling initiation of the electrochemical processes.

It is understood that even though particular components of the dressing are shown in FIGS. 2A and 2B, the electrochemical wound dressing of the present invention can also include additional and/or different adhesives, sensors, and/or other suitable electrical/mechanical components and may also exhibit a different layout.

The methods of manufacturing electrochemical wound dressings of the present invention are not particularly limited. Methods of depositing the first and second electrodes are not particularly limited and may include electron beam methods, sputtering, coating, evaporation (e.g. vacuum evaporation) and solution deposition (e.g. by using metal-filled polymer solutions). Preferably, the first and second electrodes are deposited by an electrochemical deposition and/or a solution deposition or coating process, which may be optionally followed by a heating treatment in order to further enhance the densification and uniformity of the layer. The solution deposition techniques include but are not limited to coating or printing or microdispensing methods like for example spin coating, spray coating, web printing, brush coating, dip coating, slot-die printing, ink jet printing, letter-press printing, stencil printing, screen printing, doctor blade coating, roller printing, offset lithography printing, flexographic printing, or pad printing. Preferably, the solution deposition method is an inkjet printing, stencil printing, screen printing, dispense printing or drop casting method, more preferably a stencil printing, screen printing, dispense printing or inkjet printing methods. As they enable rapid and easy fabrication of flexible and mechanically robust electrochemical wound dressings, such solution deposition techniques and/or electrochemical deposition methods are preferably also used to apply the optional conductive layers and/or electrically insulating layers.

### General Applications in Electrochemical Medical Devices or Articles

In a second embodiment, the present invention relates to the use of at least one material selected from a first material and a second material for preventing formation of or for reducing hypochlorite anions or hypochlorous acid in a medical device or article, wherein the first material is a catalyst for the electrochemical conversion of hypochlorite anions or hypochlorous acid, and wherein the second material is capable of reacting as a substitute for the precursor species leading to the generation of hypochlorite anions or hypochlorous acid and/or capable of reducing hypochlorite anions or hypochlorous acid.

The medical device or article is preferably an electrochemical medical device or article, which performs a function inside or on the surface of the human body by means of electrochemical process. Examples thereof include drug delivery apparatuses, neurostimulators, electrochemical cells, implantable or external sensors, and electrochemical wound dressings. Preferably, the electrochemical medical device or article comprises or is in contact with an aqueous solution comprising precursor species leading to the generation of hypochlorite anions or hypochlorous acid during operation of the electrochemical medical device or article (such as chloride or chlorine, for example).

In other embodiments, which involve the use in medical devices or articles which exert their function without the involvement of electrochemical processes, it may be preferable to use the second material, especially preferably a second material capable of reducing hypochlorite anions or hypochlorous acid.

The first and second materials to be used herein correspond to those defined above with respect to the first embodiment.

Overall, it will be appreciated that the preferred features of the first and second embodiments specified above may be combined in any combination, except for combinations where at least some of the features are mutually exclusive.

Once given the above disclosure, many other features, modifications, and improvements will become apparent to the skilled artisan.

### REFERENCE NUMERALS

1: substrate

2: adhesive layer

3: first electrode (anode)

4: conductive layer

5: second electrode (cathode)

6: electrically insulating layer

7: power source

8: constant current control unit

1. Electrochemical wound dressing, comprising:
first and second electrodes, an electrical power source in electrical contact with the first and the second electrode, and at least one material selected from a first material and a second material;
wherein the first material is a catalyst for the electrochemical conversion of hypochlorite anions or hypochlorous acid, and
wherein the second material is capable of reacting as a substitute for the precursor species leading to the generation of hypochlorite anions or hypochlorous acid and/or capable of reducing hypochlorite anions or hypochlorous acid.

2. The electrochemical wound dressing according to claim 1, wherein the first material is selected from a metal, a metal oxide, an organometallic complex, and mixtures thereof.

3. The electrochemical wound dressing according to claim 2, wherein the first material comprises one or more metals selected from metals from any of groups 4 to 11, preferably from any of groups 4 and 8 to 11, or alkaline earth metals.

4. The electrochemical wound dressing according to claim 3, wherein the first material comprises one or more metals selected from Mg, Ca, Ti, Mo, Mn, Fe, Ru, Co, Rh, Ir, Ni, Pd, Pt, and Cu, their oxides and complexes thereof; preferably Mg, Fe, Co, Ni and Cu, their oxides and complexes thereof.

5. The electrochemical wound dressing according to claim 4, wherein the first material comprises a cobalt porphyrin complex, preferably vitamin B12.

6. The electrochemical wound dressing according to claim 1, wherein the first material is comprised in a porous catalyst layer in contact with the first or the second electrode.

7. The electrochemical wound dressing according to claim 1, wherein the first material is comprised in a catalyst layer between the first and the second electrode.

8. The electrochemical wound dressing according to claim 6, wherein a membrane layer is provided over the catalyst layer, preventing direct contact of the catalyst layer with the wound.

9. The electrochemical wound dressing according to claim 1, wherein the wherein the second material is selected from ascorbic acid, sodium ascorbate, ascorbate polymer, methionine, lycopene, chlorogenic acid, lipoic acid, taurine, ammonium salts and thiols.

10. The electrochemical wound dressing according to claim 1, wherein the second material is comprised in a layer in contact with the electrode functioning as an anode.

11. The electrochemical wound dressing according to claim 1, further comprising a conductive polymer layer over and in contact with the first or the second electrode.

12. The electrochemical wound dressing according to claim 1, further comprising a constant current control unit between and in electrical contact with the electrical power source and the first and second electrodes.

13. The electrochemical wound dressing according to claim 1, wherein the electrical power source is a flexible battery, and wherein the electrochemical wound dressing preferably comprises a battery state indicator.

14. Use of at least one material selected from a first material and a second material for preventing formation of or for reducing hypochlorite anions or hypochlorous acid in a medical device or article,
wherein the first material is a catalyst for the electrochemical conversion of hypochlorite anions or hypochlorous acid, and
wherein the second material is capable of reacting as a substitute for the precursor species leading to the generation of hypochlorite anions or hypochlorous acid and/or capable of reducing hypochlorite anions or hypochlorous acid.

15. Use according to claim 14, wherein the medical device or article is an electrochemical device or article which comprises or is in contact with an aqueous solution comprising precursor species leading to the generation of hypochlorite anions or hypochlorous acid during its operation.
